(19) United States
(12) Patent Application Publication
(10) Pub. No.: US 2022/0235012 A1
(43) Pub. Date: Jul. 28, 2022

(54) ACID ADDITION SALT OF PYRIMETHAMINE

(71) Applicant: GLG PHARMA S.A., WROCLAW (PL)

(72) Inventors: LUKAWSZ KACZMAREK, WARSZAWA (PL); MARTA LASZCZ, WARSZAWA (PL); GRZEGORZ HUSZCZA, WARSZAWA (PL); MALGORZATA SKAZNIK, WARSZAWA (PL); MARTA ZEZULA, WARSZAWA (PL); ALEKSANDRA GROMAN, WARSZAWA (PL); ELZBIETA STOLARCZYK, WARSZAWA (PL); MAREK KUBISZEWSKI, LLÓW (PL); KINGA TRZCINSKA, PILAWA (PL); KRZYSZTOF KUZIAK, WARSZAWA (PL)

(21) Appl. No.: 17/049,524

(22) PCT Filed: Mar. 22, 2019
(86) PCT No.: PCT/PL2019/000021

§ 371 Date: Oct. 21, 2020

(30) Foreign Application Priority Data
Mar. 22, 2018 (PL) P.424999

(51) Int. Cl. C07D 239/49; C07C 309/04; A61K 9/20; A61K 9/48

(52) U.S. Cl. CPC C07D 239/49; A61K 9/48; A61K 9/20; C07C 309/04

(57) ABSTRACT

Acid addition salt of pyrimethamine (5-4-chlorophenyl)-6-ethyl-2,4-pyrimidinediamine) and methane sulfonic acid, process for its preparation and pharmaceutical compositions comprising the acid addition salt are disclosed.

### CROSS-REFERENCE TO RELATED APPLICATION

This application is the National Stage of International Application Number PCT/PL2019/000021, filed Mar. 22, 2019, which is hereby incorporated by reference herein in its entirety, including any figures, tables, nucleic acid sequences, amino acid sequences, or drawings.

### FIELD OF INVENTION

The present invention relates to a new acid addition salt of pyrimethamine, process for its preparation and the pharmaceutical compositions comprising thereof.

### BACKGROUND OF THE INVENTION

Pyrimethamine, 5-(4-chlorophenyl)-6-ethyl-2,4-pyrimidinediamine, is a dihydrofolate reductase (DHFR) inhibitor with antiparasitic properties, approved for the treatment of malaria. Its activity against Plasmodium and Toxoplasma protozoa is based on a selective inhibition of folic acid transformation, leading to the failure of the synthesis of folinic acid, which is essential in nucleic acids formation. More recently, the compound is evaluated in phase 1/11 clinical studies for the treatment of chronic lymphocytic leukemia. Preclinical studies are also under way for the treatment of autosomal dominant polycystic kidney disease and in phase I/II clinical studies for the treatment of familial amyotrophic lateral sclerosis. An orphan drug designation was assigned to the compound in the U.S. for the treatment of GM-2 gangliosidoses, including Tay-Sachs disease and Sandhoff disease and in Japan for the treatment of toxoplasmosis.

The U.S. Pat. No. 2,576,939 discloses some 2,4-diamine-5-phenyl-6-alkylpyrimidine derivatives with antimalarial properties.

Extremely low solubility of pyrimethamine in water (10 mg/L) affects adversely its bioavailability. Previous attempts to increase the pyrimethamine solubility include formation of complexes with cyclodextrin (J. Pharm. Pharmaceutical Si., 2012, 4(4), 102), low molecular weight succinoglucan dimers (Bull. Korean Chem. Soc., 2012, 33(8), 2731), or nanosuspension stabilized with polyelectrolytes (J. Bionanosci., 2010, 4(1-2), 123). There are also reports of pyrimethamine salts with levulinic (JP 46034992 B), pamoic and citrazinic (GB 986812 A) acids. Although the synthesis and crystallographic characteristics of some pyrimethamine salts with common acids, i.e. maleic, succinic, phthalic, fumaric, glutaric or formic ones was described (Crystal Growth and Design, 2002, 2/6, 631; 2003, 35, 823), their solubility in water was not presented. Some salts of pyrimethamine and inorganic acids as hydrochloric or sulfuric acids are also known in the art.

In attempts to find out the method of derivatization of pyrimethamine into more soluble and bioavailable derivative thereof, some pyrimethamine salts have been produced and their solubility in water and water mixtures with most commonly used solubilizers, like non-ionic surfactants/emulsifying agents, eg. polyoxythylene sorbitan fatty acid esters (Tween 80) or polyoxyethylene hydrogenated castor oil derivatives (Cremophor RH 40), has been tested. It was pyrimethamine and methanesulfonic acid salt that fulfilled the criteria of solubility within the broadest possible range.

### BRIEF DESCRIPTION OF THE FIGURES

FIG. 1 represents the structure (ORTEP) of the pyrimethamine methanesulfonate molecule.

FIG. 2 represents infrared absorption spectrum of pyrimethamine methanesulfonate recorded in KBr tablets.

FIG. 3 represents the X-Ray powder diffraction pattern (XRPD) in comparison with the simulated XRPD of the crystalline pyrimethamine methanesulfonate.

FIG. 4 represents the thermogravimetric analysis of the crystalline pyrimethamine methanesulfonate.

### SUMMARY OF THE INVENTION

The present invention provides methanesulfonic acid salt of 5-(4-chlorophenyl)-6-ethyl-2,4-pyrimidinediamine as a new chemical entity, referred hereinafter as pyrimethamine methanesulfonate.

Although there is a potential possibility of formation of two addition salts of methanesulfonic acid with pyrimethamine, i.e. in molar ration 1:1 or 2:1, it appeared, that without regard to reagents proportion only monomethanesulfonate of pyrimethamine (1:1) is formed. Thus, the new salt, pyrimethamine methanesulfonate according to the invention, is represented by the formula (I).

The invention also provides a process for preparation of pyrimethamine methanesulfonate represented by the formula (I) characterized in that 5-(4-chlorophenyl)-6-ethyl-2,4-pyrimidinediamine, dispersed or dissolved in organic solvent, is reacted with methanesulfonic acid.

The starting compound for the salt formation, 5-(4-chlorophenyl)-6-ethyl-2,4-pyrimidinediamine (pyrimethamine base), may be obtained according to any procedure known in the art, e.g. by the method disclosed in the specification of U.S. Pat. No. 2,576,939. Preferably, in the reaction with methanesulfonic acid, chemically pure pyrimethamine base is used, re-crystallized in polyhydroxyl alcohol, e.g. in ethylene glycol.

The salt forming reaction is carried out with the use of a slight molar excess of methanesulfonic acid in the relation to pyrimethamine base. Typically the molar ratio of methanesulfonic acid to pyrimethamine base is in a range from 1.01:1 to 1.10:1.

In the preferred embodiment of the present invention pyrimethamine methanesulfonate is prepared in a process comprising:
(i) combining 5-(4-chlorophenyl)-6-ethyl-2,4-pyrimidinediamine and methanesulfonic acid in an organic solvent,
(ii) heating the mixture at the temperature within the range from 10° C. to reflux, until the solids completely dissolves,
(iii) optionally, adding an anti-solvent and/or seeding crystals to the reaction mixture,
(iv) cooling down the post-reaction mixture to the crystallization temperature (0° C.-25° C.),
(v) crystallization and isolation of the crystalline product, and
(vi) drying the crystalline product.

The suitable organic solvents are selected from the group comprising the polar C₁-C₃ aliphatic alcohols, C₃-C₅ ketones, polyhydroxy alcohols (glycols), or the mixtures thereof. The preferred reaction solvents are ethanol, acetone or the mixture of ethylene glycol and acetone.

The precipitation of the formed crystals could be facilitated by the addition of anti-solvent of C₃-C₅ ketone type and/or the seeding crystals to the post-reaction mixture.

After cooling down the post-reaction mixture to the crystallization temperature, usually within the range from 5° C. to ambient temperature (ca. 20-25° C.), the crystalline product precipitates out. The crystals are isolated in the typical manner, for example by filtration, decantation or solvent(s) evaporation. The solvent(s) evaporation is carried out to achieve their levels commonly accepted for the pharmaceutical active ingredients and depicted in the ICH Guidelines.

Pyrimethamine methanesulfonate is obtained in the process according to the invention with a high yield of more than 70%, calculated on the starting pyrimethamine base. The crystalline pyrimethamine methanesulfonate isolated from the post-reaction mixture is distinguished by a very high chemical purity, regardless of the starting pyrimethamine base purity. Typically, the purity of pyrimethamine methanesulfonate determined by the method of Ultra-High Performance Liquid Chromatography (UH-PLC), without any further purification, exceeds 99.0%.

The crystalline pyrimethamine methanesulfonate may be additionally purified by recrystallization, if there is the need thereof.

The ¹H-NMR proton Magnetic Resonance spectrum as well as the elemental analysis confirm that the salt of the invention contains a pyrimethamine base and methanesulfonic acid in a molar ratio of 1:1.

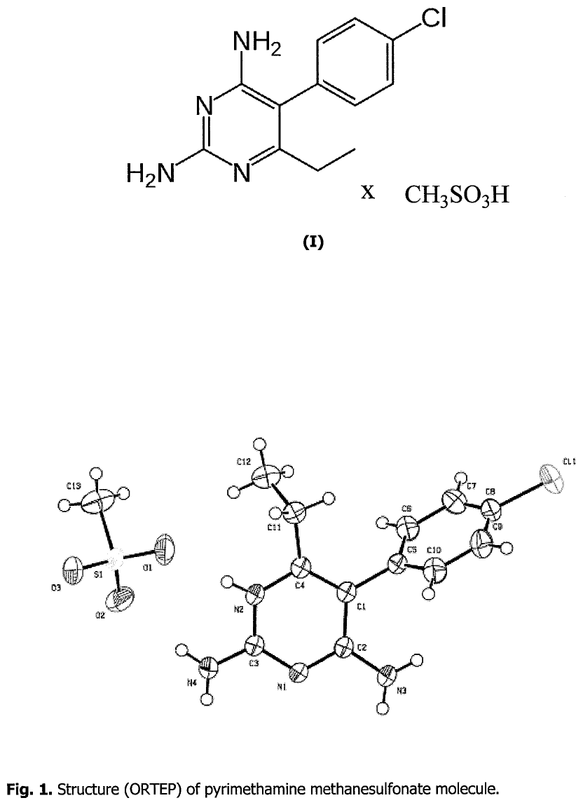

The structure of crystalline pyrimethamine methanesulfonate was elucidated by a single-crystal X-ray diffraction analysis. The molecular structure (ORTEP) of pyrimethamine methanesulfonate is presented in FIG. 1.

Pyrimethamine methanesulfonate crystallizes in the triclinic crystal system in the P-1 space group. Crystallographic data, in particular the unit cells dimensions, the volume of each cell, calculated density, and the measurement parameters are presented in Table 1.

TABLE 1
Crystallographic data and structure refinement for
the pyrimethamine methanesulfonate molecule.

| Empirical formula | C₁₂H₁₄ClN₄¹⁺•CH₃O₃S¹⁻ |
|---|---|
| Formula weight | 344.82 |
| Temperature/K | 295(2) |
| Crystal system | Triclinic |
| Space group | P-1 |
| a/Å | 8.3293(4) |
| b/Å | 8.4689(4) |
| c/Å | 11.4390(5) |
| α/° | 94.451(4) |
| β/° | 96.775(4) |
| γ/° | 94.048(4) |
| Volume/Å³ | 796.27(6) |
| Z | 2 |
| ρ_{calc}g/cm³ | 1.438 |
| μ/mm⁻¹ | 3.515 |
| F (000) | 362.4262 |
| Crystal size/mm³ | 0.38 × 0.24 × 0.24 |
| Radiation | CuKα (λ = 1.54184 Å) |
| 2Θ range for | 7.82 to 134.16 |
| data collection/° | |
| Index ranges | −8 ≤ h ≤ 9, −10 ≤ k ≤ 7, −13 ≤ l ≤ 12 |
| Reflections collected | 4818 |
| Independent reflections | 2828 [Rᵢₙₜ = 0.0192, R_{sigma} = 0.0249] |
| Data/restraints/parameters | 2827/0/217 |
| Goodness-of-fit on F² | 1.038 |
| Final R indexes | R₁ = 0.0383, wR₂ = 0.1039 |
| [I >= 2σ (I)] | |
| Final R indexes | R₁ = 0.0417, wR₂ = 0.1085 |
| [all data] | |
| Largest diff. peak/hole/eÅ⁻³ | 0.29/−0.38 |

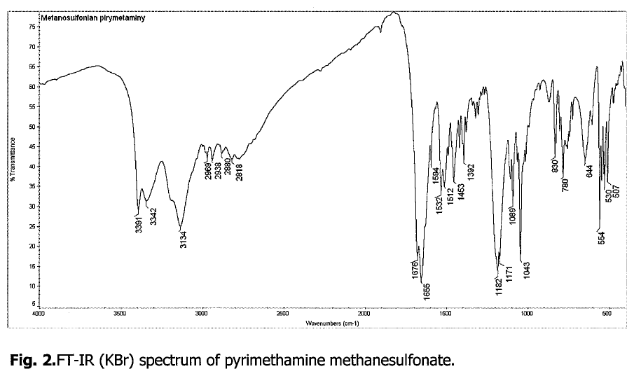

The unique infrared absorption spectrum of pyrimethamine methanesulfonate recorded from KBr pellets by the Fourier-transform Infrared Spectroscopy (FTIR) is presented in FIG. 2.

The crystalline pyrimethamine methanesulfonate is characterized by an X-Ray powder diffraction pattern (XRPD) recorded with the use of CuKα radiation source having the wavelength λ=1.54056 Å, showing the characteristic peaks presented as the relation of reflection angles 2θ (°), interplanar spacings d (Å), and relative intensities in attitude to the most intensive diffraction peak, I/I₀ (%), as depicted in Table 2:

TABLE 2
X-Ray powder diffraction of pyrimethamine methanesulfonate

| 2θ (°) | d (Å) | I/Iₘₐₓ (%) |
|---|---|---|
| 10.49 | 8.430 | 2 |
| 10.73 | 8.242 | 3 |
| 12.49 | 7.081 | 2 |
| 13.60 | 6.504 | 3 |
| 14.41 | 6.142 | 6 |
| 15.64 | 5.662 | 8 |
| 16.20 | 5.467 | 3 |
| 16.58 | 5.343 | 5 |
| 17.84 | 4.968 | 2 |
| 18.46 | 4.801 | 2 |
| 19.61 | 4.524 | 12 |
| 20.07 | 4.422 | 7 |
| 21.00 | 4.228 | 100 |
| 21.53 | 4.123 | 10 |
| 23.55 | 3.775 | 26 |
| 23.69 | 3.752 | 18 |
| 24.39 | 3.647 | 6 |
| 24.72 | 3.598 | 6 |
| 24.94 | 3.567 | 7 |
| 25.18 | 3.534 | 7 |
| 26.68 | 3.339 | 3 |
| 27.11 | 3.286 | 13 |
| 28.12 | 3.170 | 4 |
| 29.04 | 3.072 | 3 |
| 29.89 | 2.987 | 8 |

The unique infrared absorption spectrum of pyrimethamine methanesulfonate recorded from KBr pellets by the Fourier-transform Infrared Spectroscopy (FTIR) is presented in FIG. 2.

The crystalline pyrimethamine methanesulfonate is characterized by an X-Ray powder diffraction pattern (XRPD) recorded with the use of CuKα radiation source having the wavelength λ=1.54056 Å, showing the characteristic peaks presented as the relation of reflection angles 2θ (°), interplanar spacings d (Å), and relative intensities in attitude to the most intensive diffraction peak, I/I₀ (%), as depicted in Table 2:

TABLE 2
X-Ray powder diffraction of pyrimethamine methanesulfonate

| 2θ (°) | d (Å) | I/Iₘₐₓ (%) |
|---|---|---|
| 7.84 | 11.275 | 30 |
| 10.49 | 8.430 | 2 |
| 10.73 | 8.242 | 3 |
| 12.49 | 7.081 | 2 |
| 13.60 | 6.504 | 3 |
| 14.41 | 6.142 | 6 |
| 15.64 | 5.662 | 8 |
| 16.20 | 5.467 | 3 |
| 16.58 | 5.343 | 5 |
| 17.84 | 4.968 | 2 |
| 18.46 | 4.801 | 2 |
| 19.61 | 4.524 | 12 |
| 20.07 | 4.422 | 7 |
| 21.00 | 4.228 | 100 |
| 21.53 | 4.123 | 10 |
| 23.55 | 3.775 | 26 |
| 23.69 | 3.752 | 18 |
| 24.39 | 3.647 | 6 |
| 24.72 | 3.598 | 6 |
| 24.94 | 3.567 | 7 |
| 25.18 | 3.534 | 7 |
| 26.68 | 3.339 | 3 |
| 27.11 | 3.286 | 13 |
| 28.12 | 3.170 | 4 |
| 29.04 | 3.072 | 3 |
| 29.89 | 2.987 | 8 |

TABLE 2
X-Ray powder diffraction of pyrimethamine methanesulfonate

| 2θ (°) | d (Å) | I/Iₘₐₓ (%) |
|---|---|---|
| 7.84 | 11.275 | 30 |
| 30.22 | 2.955 | 5 |
| 31.32 | 2.853 | 2 |
| 31.88 | 2.805 | 3 |
| 33.48 | 2.674 | 2 |
| 34.58 | 2.592 | 4 |
| 35.14 | 2.552 | 2 |

The experimental X-Ray powder diffractogram of pyrimethamine methanesulfonate (lower pattern), recorded with the use of CuKα (λ=1.54056 Å), is essentially consistent with the simulated XRPD (upper pattern).

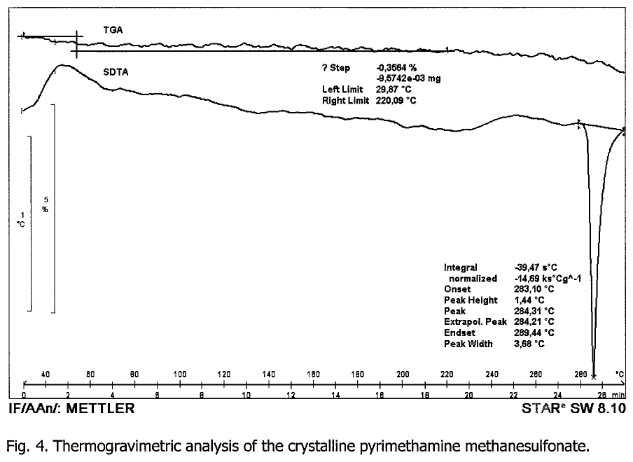

The melting point of pyrimethamine methanesulfonate was determined as the onset temperature being the intersection of tangent lines to baseline and the leading edge of melting peak from the single differential thermal analysis (SDTA) curve from thermogravimetric analysis (TGA) (FIG. 4). The SDTA signal shows the melting of salt at Tₒₙₛₑₜ=283.10° C.

The mass loss (Δm/m, %), determined from TGA curve within the temperature range from 30 to 220° C., is 0.36% indicating non-solvate form of the pyrimethamine methanesulfonate salt.

In contrary to other salts of pyrimethamine, pyrimethamine methanesulfonate is freely soluble in water even at ambient temperature, without any necessity of surfactant and/or emulsifier addition.

It can be anticipated that pyrimethamine methanesulfonate will possess the same pharmacological properties as pyrimethamine base.

The new pyrimethamine methanesulfonate salt is well tolerated and pharmaceutically accepted (see, Handbook of Pharmaceutical Salts, ed. P. H. Stahl. C. G. Wermuth, Verlag Helvetica Chimica Acta, 2002). Due to its advantageous physicochemical and toxicological properties, it may be used in the therapy and prevention of different diseases in humans.

For the therapeutic use, the active substance pyrimethamine methanesulfonate may be administered to the patient per se, or as a pharmaceutical composition comprising therapeutically effective amount of the active substance together with at least one pharmaceutically acceptable carrier and/or excipients.

Accordingly, the present invention also provides a pharmaceutical composition comprising pyrimethamine methanesulfonate as the active ingredient which may be administered to a patient in a need for treatment in an appropriate pharmaceutical dosage form, dependent on the mode of administration. The orally or parenterally administrable pharmaceutical dosage forms are preferred.

The active substance dose selection and the treatment regimens depend on the disease progression, age, body weight and condition of the patient, and may be determined by a skilled person basing on the known treatment and prophylaxis regimes appropriate for this kind of diseases.

The appropriate daily dose of pyrimethamine methanesulfonate may be administered to the patient either as a single daily dose or in 2 or more divided doses, as monotherapy or in combination with other therapeutics. The components of such combinations may be administered to the patient in the form of one combined fixed-dosage pharmaceutical formulation or in separate formulations one after the other in order and time intervals established by a skilled person.

The pharmaceutical composition according to the present invention may be administered in the pharmaceutical form well-known to those skilled in the art. See: e.g. Remington's Pharmaceutical Sciences, 18^{th} ed., red. A. R. Gennaro, Mack Publ. Co., 1990, Easton, Pa.

The pharmaceutical compositions may be adopted for oral administration, although compositions for administration by other routes, such as parenteral, are also envisaged.

The pharmaceutical oral dosage forms comprise solid dosage forms, such as tablets, coated tablets, powders, granules, pellets or capsules; and liquid dosage forms, such as suspensions, elixirs, solutions and syrups. In addition to the active substance they contain pharmaceutically acceptable fillers and/or excipients. The pharmaceutically acceptable fillers and/or excipients are the substances or mixtures thereof generally known in the art as not exerting their own pharmacological effect.

The suitable fillers for use in the solid dosage forms for the conventional release of the active substance include starch, lactose, microcrystalline cellulose, saccharose, sorbitol, talc, mannitol, mono- or dibasic calcium phosphate, pregelatinized starch, glycine and others.

The solid oral dosage forms may further contain excipients facilitating the manufacturing process and imparting required physico-mechanical properties to the finished dosage form.

Further excipients may be selected from disintegrants, such as starch and starch derivates, crosscarmellose sodium, microcrystalline cellulose, crosslinked polyvinylpyrrolidone, starch sodium glycolate or other products based on crosslinked polymer; binders, such as polyvinylpyrrolidone, gelatin, natural and synthetic gums, cellulose derivative, e.g. hydroxypropyl methylcellulose, hydroxyethyl cellulose, hydroxypropyl cellulose; lubricants, such as sodium lauryl sulphate; glidants, such as colloidal silicon dioxide, stearic acid, magnesium stearate, talc, fumaric acid and others.

The tablets optionally may be coated as described for example in Pharmaceutical Dosage Forms and Drug Delivery Systems, H. C. Ansel, L V. Allen, N. G. Popovich, VII^{th} ed. (1999), Lippincott Williams & Wilkins. The coating formulations preferably contain film coating substance selected to provide the dissolution or fragmentation of the coating in the desired gastrointestinal tract section, together with the pharmaceutical excipients, such as plasticizers, fillers, opacifiers, colourants and polishing agents. The film coating substances are preferably polymers such as cellulose derivatives, acrylic polymers and copolymers, high molecular weight polyethylene glycols, polyvinylpyrrolidone, polyvinyl alcohol and others. Suitable plasticizers can be polyols, such as glycerol; organic esters such as phthalates, sebacates or citrates, and others.

Administration of the pharmaceutical compositions comprising pyrimethamine methanesulfonate in the parenteral dosage form, e.g. for intravenous, subcutaneous or intramuscular administration, may also be considered. The parenteral compositions comprise sterile water, water-organic and non-water solutions and suspensions; lyophylisates and tablets suitable for reconstitution ex tempore. For liquid formulations suspending agents providing uniform active substance distribution in the liquid phase, such as polysorbates, lecithin, polyoxyethylene and polyoxypropylene copolymers; peptizers, such as phosphates, polyphosphates and citrates, water-soluble polymers, such as carboxymethyl cellulose, methyl cellulose, polyvinylpyrrolidone, hydrogenated oils, gums or gelatin, may be applied.

The parenteral formulations may further contain pharmaceutically acceptable additives, such as solubilizers, preservatives, pH adjusting agents, buffers and tonicity agents.

The present invention provides stable crystalline pyrimethamine methanesulfonate salt distinguished by high solubility in aqueous media. The invention further provides efficient, reproducible manufacturing process for high chromatographic purity pyrimethamine methanesulfonate in the crystalline form.

The present invention is further illustrated by the following, non-limiting, examples.

### EXAMPLES

### Analytical Methods

The UHPLC Method for Determination of Chemical Purity

The determination of chemical purity was performed using an ultra-high performance liquid chromatograph (UHPLC, DionexUltiMate300RS) with a spectrophotometric detector (DAD 3000 RS, Dionex Softron GmbH). The chromatographic separation was achieved with the use of an Acquity UPLC CSH Phenyl-hexyl, 100×2.1 mm, 1.7 μm (Waters) reversed phase analytical column, under the following conditions:
Eluent A: aqueous 0.01% H₃PO₄ solution (v/v)
Eluent B: ACN
Flow rate: 0.5 mL/min
Gradient elution parameters:

| time [min] | eluent A [%] | eluent B [%] |
|---|---|---|
| 0.0 | 95 | 5 |
| 7.0 | 5 | 95 |
| 7.5 | 5 | 95 |
| 8.0 | 95 | 5 |
| 12.0 | 95 | 5 |

Column temperature: 30° C.

The HPLC Method for Methanesulfonic Acid Assay Determination

The assay determination of methanesulfonic acid was performed using a high performance liquid chromatograph (UHPLC, DionexUltiMate300RS) with a charged aerosol detector (Corona CAD, Thermo Scientific). The chromatographic separation was achieved with the use of a Synergy Fusion RP, 150×4.6 mm, 4.0 μm (Phenomenex) reversed phase analytical column, at the following conditions:
Eluent A: aqueous 0.1% CH₃COOH solution (v/v)
Eluent B: MeOH
Flow rate: 0.8 mL/min
Gradient elution parameters:

| time [min] | eluent A [%] | eluent B [%] |
|---|---|---|
| 0.0 | 95 | 5 |
| 4.0 | 75 | 25 |
| 6.0 | 25 | 75 |
| 8.0 | 25 | 75 |
| 8.2 | 95 | 5 |
| 12.0 | 95 | 5 |

Column temperature: 30° C.

FT-IR

The infrared absorption spectra were recorded from KBr pellets on the Nicolet iS10 (Thermo Scientific) spectrometer with Fourier transform in the range from 4000 to 400 cm⁻¹ with the spectral resolution of 4 cm⁻¹.

¹H-NMR

The magnetic nuclear resonance ¹H-NMR spectra were recorded on the Bruker Avance 500 MHz spectrometer.

Thermogravimetric Analysis

The TGA measurement was performed by means of the TGA/SDTA851 cell (Mettler Toledo). About 5 mg of the studied sample was weighed into a standard aluminium pan (40 μL). The pan was hermetically sealed and perforated before the measurement. The sample was heated from 30 to 300° C. at 10° C./min in the nitrogen atmosphere. The measurement was blank curve corrected.

XRPD and Single Crystal Measurements

The X-Ray powder diffraction (XRPD) patterns were recorded on the MiniFlex diffractometer (Rigaku Corporation) with CuKα1 radiation (λ=1.54056 Å) and a scintillator detector. The sample was pressed on a glass plate. The instrument was operated in the 2θ range from 3 to 40° with the scanning speed of 0.5°/min and the θ/2θ axis step angle of 0.02°. Measurement temperature: room temperature.
Obtained diffraction patterns were worked up and handled using PDXL2 software (Rigaku Corporation).
Crystal structure was analyzed using the mono-crystalline diffractometer Agilent Technologies SuperNova Dual Source with the CuKα radiation.

### Example 1

Pyrimethamine (30 g) (99.83% purity, containing 0.16% of impurity with R_{f}=2.53) was crystallized in ethylene glycol (3×60 mL). The obtained crystals were refluxed in ethanol (50 mL), filtered off and dried under reduced pressure at 60° C. This afforded 25 g of pyrimethamine, containing 0.09% of R_{f}=2.53 impurity. The obtained product (24.9 g, 0.1 mol) was suspended in acetone (250 mL) and methanesulfonic acid (24.0 g, 0.25 mol) was added to the suspension. The reaction was stirred at rt for 60 min., the white precipitate was then collected and washed with acetone (500 mL). The product was suspended in ethanol (250 mL) and refluxed with stirring for 15 min. Then, the suspension was cooled to rt, the precipitate was collected, washed with ethanol (250 mL) and dried at 60° C. under reduced pressure (20 mm Hg). The product was obtained as white crystals, with yield 24.0 g (70%). The content of acid in the salt, found by potentiometric titration: 28.15% (calcd.: 27.87%)

¹H NMR (500 MHz, DMSO-d₆) δ (ppm): 1.01 (t (J==7.5 Hz). 3H. H-8); 2.18 (k (J=7.5 Hz). 2H. H-7); 2.47 (s. 3H. H—CH₃ salt); 6.87 (s. 1H. H-14); 7.29 (d (J=8.5 Hz). 2H. H-10); 7.54 (d (J=8.5 Hz), 2H, H-11); 7.76 (bs. 2H, H-13); 8.12 (s, 1H, H-14);

¹³C NMR (125 MHz, DMSO-d₆) δ (ppm): 12.54 (C-8); 23.55 (C-7); 39.73 (C—CH₃ salt); 107.18 (C-5); 129.44 (C-11); 130.06 (C-9); 132.48 (C-10); 133.59 (C-12); 154.46 (C-6); 154.76 (C-2); 164.11 (C-4).

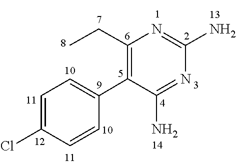 [SMILES: CCc1nc(N)nc(N)c1-c1ccc(Cl)cc1]

MS (45.43%—C; 4.80%—H; 16.37%—N; 9.25%—S; 10.37%—Cl; calc.: 45.28%—C; 4.97%—H; 16.25%—N; 9.30%—S; 10.28%—Cl)

### Example 2

Pyrimethamine (24.9 g, 0.1 mol) was dissolved in hot ethylene glycol (100 mL), methanesulfonic acid (24.0 g, 0.25 mol) was added to the solution and the reaction mixture was cooled to it. Then, acetone (200 mL) was added and the mixture was left for 12 hrs at rt. The precipitate was collected, washed with acetone (50 mL) and dried at 60° C. under reduced pressure (20 mm Hg). The product was obtained as white crystals, with yield 18.0 g (52%). The content of acid in the salt, found by potentiometric titration: 28.20% (calc.: 27.87%)

### Example 3

Pyrimethamine (249 g, 1 mol) was suspended in ethanol 96% (1 L) and methanesulfonic acid (240 g=162 mL, 2.5 mol) was added. The reaction mixture was stirred at rt for 60 min. and then under reflux for 30 min. After cooling the white precipitate was collected and washed with ethanol (500 mL). The product was suspended in ethanol (1 L) and refluxed with stirring for 15 min. The mixture was then cooled to rt, the precipitate collected and washed by turns with ethanol (200 mL), acetone (IL) and again with ethanol (200 mL). The product was dried at 60° C. under reduced pressure (20 mm Hg) to afford white crystals with yield 278 g (81%). The content of acid: 27.92% (calc.: 27.87%).

### Example 4

### Solubility of Pyrimethamine Salts

The solubility of pyrimethamine salts with different organic and mineral acids was evaluated according to the general recommendations described in Ph. Eur. 9.2 for the pharmaceutical active ingredients. The “solubility” ° according to Ph. Eur. 9.2, is the approximate volume of solvent in millilitres per gram of solute. The solubility of the substance is classified in seven categories, from very soluble (less than 1 mL per 1 g) to practically insoluble more than 10,000 mL per 1 g). Due to the extremely low solubility of some salts, however, determination of exact value according to Ph. Eur. was not possible. Thus, the solubility was determined according to own method at two temperatures, 20° C. and 60° C. The results are presented in the Table 3 below.

TABLE 3
Solubility of pyrimethamine salts

| | | | | | 10% | | 10% | | Solubility in |
|---|---|---|---|---|---|---|---|---|---|
| | | Concentration | H₂0 | | Tween 80 | | Cremophor RH 40 | | terms of |
| No. | Salt | [mg/g] | 20° C. | 60° C. | 20° C. | 60° C. | 20° C. | 60° C. | Ph. Eur. 9.2*** |
| 1 | Formate | 10 | − | − | − | +/+ | − | +/+ | Slightly soluble |
| 2 | Methanesulfonate | 15 | +/+ | | +/+ | | +/+ | | Sparingly soluble |
| 3 | Sulfate | 15 | − | +/− | +/+(−) | | +/+(−) | | Sparingly soluble |
| 4 | Tartrate | 15 | +/+(−) | | +/+ | | +/+ | | Sparingly soluble |
| 5 | Oxalate | 15 | | +/− | − | − | − | − | Slightly soluble |
| 6 | Acetate* | 15 | − | − | − | − | − | − | Less than |
| | | | | | | | | | slightly soluble |
| 7 | Lactate* | 15 | − | − | − | − | − | − | Less than |
| | | | | | | | | | slightly soluble |
| 8 | Hydrochloride** | 15 | − | − | − | − | − | − | Very slightly |
| | | | | | | | | | soluble |

| “−” - insoluble; “+” - soluble; “/+” - doesn't precipitate after cooling; “/+(−)” - doesn't precipitate after cooling, but crystallizes during storage | |
|---|---|
| *due to the solubility lower than expected, determination of exact value according to Ph. Eur. was not possible | |
| **literature data | |
| ***Solubility according Ph. Eur. 9.2: | |
| Descriptive terms | Approximate volume of solvent [ml/l g] |
| very soluble | <1 |
| freely soluble | from 1 to 10 |
| soluble | from 10 to 30 |
| sparingly soluble | from 30 to 100 |
| slightly soluble | from 100 to 1 000 |
| very slightly soluble | from 1 000 to 10 000 |
| practically insoluble | >10 000 |

### Example 5

### Parenteral Formulation

| | | | Concentration |
|---|---|---|---|
| No. | Function | Substance name | (%) |
| 1 | API | Pyrimethamine | 1.5-2.5 |
| | | methanesulfonate | |
| 2 | Solubilizer | Tween 80; Cremophor RH 40; | 0-10% |
| | | Kolliphor HS 15 | |
| 3 | Buffer | | qs. pH 6-8 |
| 4 | Tonicity agent | Mannitol, sorbitol, glycerin, | 0-5 |
| | | NaCl | |
| 5 | | | |

### Example 6

### Oral Solution Formulation

| | | | Concentration |
|---|---|---|---|
| No. | Function | Substance name | (%) |
| 1 | API | Pyrimethamine | 1.5-2.5 |
| | | methanesulfonate | |
| 2 | Solubilizer | Tween 80; Cremophor RH 40; | 0-10% |
| | | Kolliphpr HS 15 | |
| 3 | Co-solvent | Propylene glycol, etanol, | 0-50% |
| | | isopropanol, glycerin, PEG (300, | |
| | | 400); 2-pyrrolidone | |
| 4 | Solvent | Water | Ad. 100 |

### Example 7

### Solid Oral Dosage Form (Tablet, Capsule)

| | | | Concentration |
|---|---|---|---|
| No. | Function | Substance name | (%) |
| 1 | API | Pyrimethamine | 1.5-2.5 |
| | | methanesulfonate | |
| 2 | Filler | Microcrystalline cellulose, | 10-90 |
| | | lactose, corn starch, mannitol | |
| 3 | Binder | HPMC, PVP, acacia gum, | 0-5 |
| | | gelatine | |
| 4 | Disintegrant | Croscarmelose sodium, sodium | 0-8 |
| | | starch glycolate, crospovidone | |
| 5 | Lubricant, | Magnesium stearate, magnesium | 0.1-5 |
| | glidant | stearyl fumarate, talc | |

We claim:

1. An acid addition salt of 5-(4-chlorophenyl)-6-ethyl-2,4-pyrimidynediamine and methanesulfonic acid in the molar proportion 1:1.

2. The acid addition salt according to claim 1 in the crystalline form characterized by the unit cell parameters:

| | |
|---|---|
| a [Å] | 8.3293(4) |
| b [Å] | 8.4689(4) |
| c [Å] | 11.4390(5) |
| α [°] | 94.451(4) |
| β [°] | 96.775(4) |
| γ [°] | 94.048(4) |

3. The acid addition salt according to claim 1, characterized by an X-ray powder diffraction pattern (XRPD) recorded with the use of CuKα radiation source having the wavelength λ=1.54056 Å, showing the characteristic peaks presented as the relation of reflection angles 2θ (°), interplanar spacings d (Å), and relative intensities in attitude to the most intensive diffraction peak, I/I₀ (%):

| 2θ (°) | d (Å) | I/Iₘₐₓ (%) |
|---|---|---|
| 7.84 | 11.275 | 30 |
| 10.49 | 8.430 | 2 |
| 10.73 | 8.242 | 3 |
| 12.49 | 7.081 | 2 |
| 13.60 | 6.504 | 3 |
| 14.41 | 6.142 | 6 |
| 15.64 | 5.662 | 8 |
| 16.20 | 5.467 | 3 |
| 16.58 | 5.343 | 5 |
| 17.84 | 4.968 | 2 |
| 18.46 | 4.801 | 2 |
| 19.61 | 4.524 | 12 |
| 20.07 | 4.422 | 7 |
| 21.00 | 4.228 | 100 |
| 21.53 | 4.123 | 10 |
| 23.55 | 3.775 | 26 |
| 23.69 | 3.752 | 18 |
| 24.39 | 3.647 | 6 |
| 24.72 | 3.598 | 6 |
| 24.94 | 3.567 | 7 |
| 25.18 | 3.534 | 7 |
| 26.68 | 3.339 | 3 |
| 27.11 | 3.286 | 13 |
| 28.12 | 3.170 | 4 |
| 29.04 | 3.072 | 3 |
| 29.89 | 2.987 | 8 |
| 30.22 | 2.955 | 5 |
| 31.32 | 2.853 | 2 |
| 31.88 | 2.805 | 3 |
| 33.48 | 2.674 | 2 |
| 34.58 | 2.592 | 4 |
| 35.14 | 2.552 | 2 |

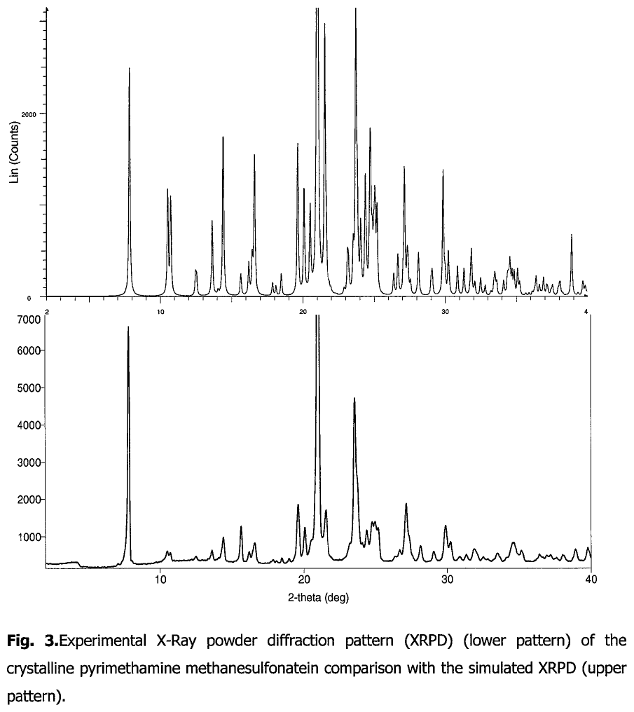

4. The acid addition salt according to claim 2, characterized by the experimental X-ray powder diffraction pattern (XRPD) substantially as depicted in FIG. 3 (lower pattern).

5. The acid addition salt according to claim 2, characterized by the melting point determined as the onset temperature from the single differential thermal analysis (SDTA) curve from thermogravimetric analysis (TGA), Tₒₙₛₑₜ=283.10° C., and the loss on drying <1.0%.

6. A process for preparation of pyrimethamine methanesulfonate, characterized in that 5-(4-chlorophenyl)-6-ethyl-2,4-pyrimidinediamine (pyrimethamine base), dispersed or dissolved in an organic solvent, is reacted with the slight molar excess of methanesulfonic acid.

7. The process according to claim 6, wherein the process is carried out with the use of the molar ratio of methanesulfonic acid to pyrimethamine base is within a range from 1.01:1 to 1.10:1.

8. The process according to claim 6, wherein the organic solvent is selected from the group comprising the polar C₁-C₃ aliphatic alcohols, C₃-C₅ ketones, polyhydroxy alcohols (glycols), or the mixtures thereof.

9. The process according to claim 6, wherein the organic solvents are ethanol, acetone or the mixture of ethylene glycol and acetone.

10. The process according to claim 6, comprising the steps of:
(i) combining 5-(4-chlorophenyl)-6-ethyl-2,4-pyrimidinediamine and methanesulfonic acid in an organic solvent,
(ii) heating the mixture at the temperature within the range from 10° C. to reflux, until the solids completely dissolves,
(iii) optionally, adding an anti-solvent and/or seeding crystals to the reaction mixture,
(iv) cooling down the post-reaction mixture to the crystallization temperature (0° C.-25° C.),
(v) crystallization and isolation of the crystalline product, and
(vi) drying the crystalline product.

11. The process according to claim 6, wherein the anti-solvent is C₃-C₅ ketone.

12. The process according to claim 6, wherein in step a) chemically pure pyrimethamine base is used after re-crystallization in polyhydroxyl alcohol, eg. ethylene glycol.

13. A pharmaceutical composition comprising a therapeutically effective amount of pyrimethamine methanesulfonate of formula (I) together with at least one pharmaceutically acceptable carrier and/or excipients.

14. The pharmaceutical composition according to claim 13, in a parenteral dosage form.

15. The pharmaceutical composition according to claim 13, in an oral dosage form.

16. The pharmaceutical composition according to claim 15, in an oral solution form.

17. The pharmaceutical composition according to claim 13, in a tablet form.

18. The pharmaceutical composition according to claim 13, in a capsule form.
